(19) United States
(12) Patent Application Publication
(10) Pub. No.: US 2025/0164652 A1
(43) Pub. Date: May 22, 2025

(54) RADIATION DETECTOR CONTAINING A CONNECTOR LOCATED WITHIN AN OPENING IN A SUPPORTING SUBSTRATE

(71) Applicant: REDLEN TECHNOLOGIES, INC., Saanichton (CA)

(72) Inventors: Eric KYFIUK, Victoria (CA); Michael AYUKAWA, Victoria (CA); Bernard HARRIS, Vancouver (CA); Krzysztof INIEWSKI, Port Moody (CA)

(21) Appl. No.: 18/943,129

(22) Filed: Nov. 11, 2024

Related U.S. Application Data
(60) Provisional application No. 63/601,108, filed on Nov. 20, 2023.

(51) Int. Cl. G01T 1/24; A61B 6/42

(52) U.S. Cl. CPC G01T 1/247; A61B 6/4233; G01T 1/244

(57) ABSTRACT

A detector structure includes a supporting substrate having an opening therethrough that is laterally surrounded on all sides by the supporting substrate, a carrier board located over a front side of the supporting substrate, the carrier board including interconnect structures electrically extending between the front side and a back side of the carrier board; at least one SIC located over the carrier board, the at least one ASIC including signal processing channel circuitry, at least one radiation sensor located over a front side of the at least one ASIC, and a connector located within the opening in the supporting substrate, the connector is electrically coupled to the interconnect structures on the back side of the carrier board. Further embodiments include detector modules including a plurality of above-described detector structures, a module circuit board coupled to the connectors by cables, and a heat sink.

### FIELD

The present disclosure relates generally to radiation detectors, and more specifically to radiation detectors containing a connector located within the opening in a supporting substrate.

### BACKGROUND

Room temperature pixelated radiation detectors made of semiconductors, such as cadmium zinc telluride (Cd₁₋ₓZnₓTe where 0<x<1, or “CZT”), are gaining popularity for use in medical and non-medical imaging. These applications use the high energy resolution and sensitivity of the radiation detectors.

### SUMMARY

According to an aspect of the present disclosure, a detector structure includes a supporting substrate having an opening therethrough that is laterally surrounded on all sides by the supporting substrate, a carrier board located over a front side of the supporting substrate, the carrier board including interconnect structures electrically extending between the front side and a back side of the carrier board; at least one ASIC located over the carrier board, the at least one ASIC including signal processing channel circuitry, at least one radiation sensor located over a front side of the at least one ASIC, and a connector located within the opening in the supporting substrate, the connector is electrically coupled to the interconnect structures on the back side of the carrier board.

Further embodiments include detector modules including a plurality of above-described detector structures, a module circuit board coupled to the connectors by cables, and a heat sink.

Another embodiment includes a method of fabricating a detector module, comprising: mounting a column of radiation detector units over a front side of a frame bar comprising a plurality of slots, wherein each radiation detector unit comprises a carrier board, at least one application specific integrated circuit (ASIC) located over a front side of the carrier board, and at least one radiation sensor located over the at least one ASIC, and a connector electrically coupled to interconnect structures on the back side of the carrier board; providing a module circuit board and a heat sink extending away from a rear side of the frame bar; and connecting a plurality of cables between the connector of each radiation detector unit and the module circuit board through the plurality of slots in the frame bar.

### BRIEF DESCRIPTION OF THE DRAWINGS

FIG. 1A is a functional block diagram of an X-ray imaging system in accordance with various embodiments of the present disclosure.

FIG. 1B is a schematically illustration of an application specific integrated circuit (ASIC) configured to count X-ray photons detected in each pixel detector within a set of energy bins according to various embodiments of the present disclosure.

FIG. 2A is a rear perspective view of a detector array for a computed tomography (CT) X-ray imaging system according to various embodiments of the present disclosure.

FIG. 2B is a perspective view of a CT X-ray imaging system illustrating the orientation of the detector array with respect to an X-ray source and a patient being imaged according to various embodiments of the present disclosure.

FIG. 3 is a vertical cross-sectional view of a radiation detector unit according to one embodiment of the present disclosure.

FIG. 4A is a top perspective view of a radiation detector unit according to various embodiments of the present disclosure.

FIG. 4B is a bottom perspective view of the radiation detector unit of FIG. 4A.

FIG. 4C is a side cross-section view of the radiation detector unit of FIGS. 4A and 4B.

FIG. 4D is a bottom view of the radiation detector unit of FIGS. 4A-4C.

FIG. 5 is a side cross-section view of a radiation detector unit according to another embodiment of the present disclosure.

FIG. 6 is a side cross-section view of a radiation detector unit including two connectors according to another embodiment of the present disclosure.

FIG. 7 is a perspective view illustrating a plurality of radiation detector units mounted on a frame bar according to various embodiments of the present disclosure.

FIG. 8A is a perspective view of a detector module according to an embodiment of the present disclosure.

FIG. 8B is a side cross-section view of the detector module of FIG. 8A.

FIG. 8C is a perspective cross-section view of the detector module of FIGS. 8A and 8B.

FIG. 9A is a side cross-section view of a portion of a detector module according to another embodiment of the present disclosure.

FIG. 9B is a perspective view of the detector module of FIG. 9A.

FIG. 10A is a side cross-section view of a portion of a detector module according to another embodiment of the present disclosure.

FIG. 10B is side cross-section view of a variant of the detector module of FIG. 10A according to another embodiment of the present disclosure.

FIG. 11 is a side cross-section view of a portion of a detector module according to yet another embodiment of the present disclosure.

FIG. 12A is a side cross-section view of a portion of a detector module according to another embodiment of the present disclosure.

FIG. 12B is a perspective view of a portion of the detector module of FIG. 12A.

FIG. 13A is a perspective view of a detector module according to another embodiment of the present disclosure.

FIG. 13B is a side cross-section view of a portion of the detector module of FIG. 13A.

### DETAILED DESCRIPTION

Embodiments of the present disclosure provide detector arrays for ionizing radiation, the various aspects of which are described herein with reference to the drawings.

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims. Any reference to claim elements in the singular, for example, using the articles “a,” “an,” or “the” is not to be construed as limiting the element to the singular. The terms “example,” “exemplary,” or any term of the like are used herein to mean serving as an example, instance, or illustration. Any implementation described herein as an “example” is not necessarily to be construed as preferred or advantageous over another implementation. The drawings are not drawn to scale. Multiple instances of an element may be duplicated where a single instance of the element is illustrated, unless absence of duplication of elements is expressly described or clearly indicated otherwise.

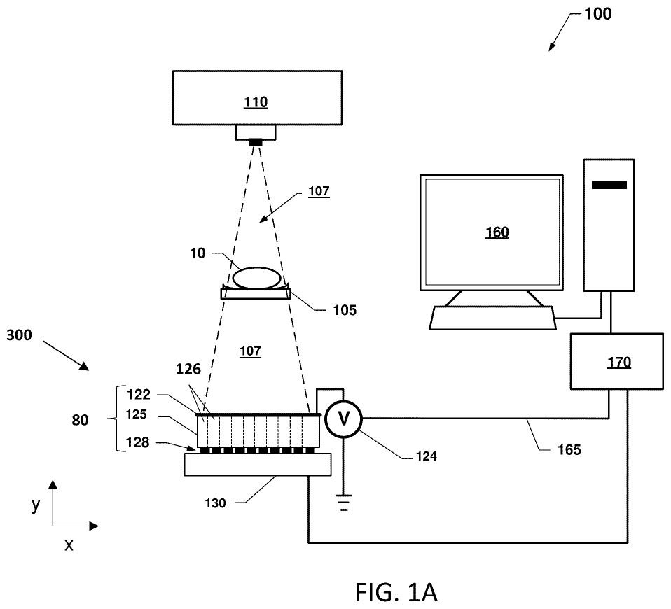

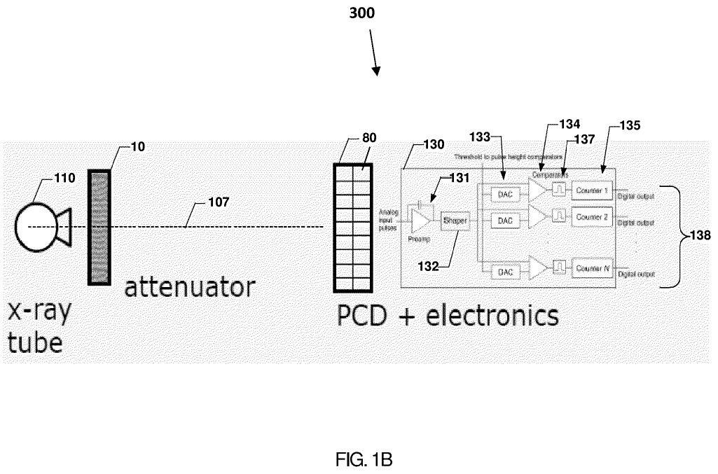

FIG. 1A is a functional block diagram of an X-ray imaging system 100 in accordance with various embodiments. The X-ray imaging system 100 may include an X-ray source 110 (i.e., a source of ionizing radiation), and an energy discriminating photon counting radiation detector array 300. The X-ray imaging system 100 may additionally include a patient support structure 105, such as a table or frame, which may rest on the floor and may support an object 10 to be scanned. In some embodiments, the object 10 may be a biologic subject (i.e., a human or animal patient). The support structure 105 may be stationary (i.e., non-moving) or may be configured to move relative to other elements of the X-ray imaging system 100, such as the X-ray source. For example, in a non-destructive testing imaging system, the support structure 105 may comprise a moving belt or web which supports the object 10. The object 10 may laterally translated by the moving belt or web past a stationary X-ray source 110, while the stationary detector array 300 is located under the belt or web. The object 10 may comprise food, baggage, manufactured products, or any other object that is subject to non-destructive testing to determine the object's composition (e.g., whether the food contains impurities), contents (e.g., contents of baggage), and/or defects (e.g., defects in manufactured products).

The X-ray source 110 may be mounted to a gantry (e.g., for CT imaging) or another support, and may move or remain stationary (e.g., in non-destructive testing) relative to the object 10. The X-ray source 110 is configured to deliver ionizing radiation to the radiation detector array 300 by emitting an X-ray beam 107 toward the object 10 and the radiation detector array 300. After the X-ray beam 107 is attenuated by the object 10, the beam of radiation 107 is received by the radiation detector array 300.

The radiation detector array 300 may include one or more radiation sensors 80 coupled to detector read-out circuitry 130. Each radiation sensor 80 may be controlled by a high voltage bias power supply 124 that selectively creates an electric field between an anode 128 and cathode 122 pair coupled thereto. In one embodiment, each radiation sensor 80 includes a plurality of anodes 128 (e.g., one anode per pixel) and one common cathode 122 electrically connected to the power supply 124 and facing the X-ray source 110. Each radiation sensor 80 may include a detector material 125, such as a semiconductor material disposed between the anode 128 and cathode 122 and thus configured to be exposed to the electrical field therebetween. The semiconductor material may comprise any suitable semiconductor material for detecting X-ray radiation disposed between the anode 128 and cathode 122 and thus configured to be exposed to the electrical field therebetween. In various embodiments, the semiconductor material of the radiation sensor(s) 80 may comprise a II-VI semiconductor material, such as cadmium telluride, cadmium zinc telluride (i.e., CdZnTe or “CZT”), cadmium selenide telluride, and cadmium zinc selenide telluride. Other suitable semiconductor materials are within the contemplated scope of disclosure.

The detector read-out circuitry may include one or more application specific integrated circuits (ASICs) 130. Each ASIC 130 may be coupled to one or more radiation sensors 80 and may receive signals (e.g., charge or current) from the anodes 128 of the radiation sensor(s) 80. Each ASIC 130 may be configured to provide data to and by controlled by a control unit 170. Each of the radiation sensors 80 may be segmented or configured into a large number of small “pixel” detectors 126. In various embodiments, the pixel detectors 126 of the radiation sensors 80 and the ASIC(s) 130 are configured to output data that includes counts of photons detected in each pixel detector 126 in each of a number of energy bins. Thus, radiation detector arrays 300 of various embodiments may provide both two-dimensional detection information regarding where photons were detected, thereby providing image information, and measurements of the energy of the detected X-ray photons. A radiation detector array 300 that is capable of measuring the energy of the X-ray photons impinging on the array 300 may be referred to as an energy-discriminating radiation detector array 300.

The control unit 170 may be configured to synchronize the X-ray source 110, the read-out ASIC(s) 130, and the high voltage bias power supply 124. The control unit 170 may be coupled to and operated from a computing device 160. Alternatively, the computing device 160 and the control unit 170 may be integrated together as one device.

In some embodiments, the X-ray imaging system 100 may be a computed tomography (CT) imaging system. The CT imaging system 100 may include a gantry (not shown in FIG. 1A), which may include a moving part, such as a circular, rotating frame with the X-ray source 110 mounted on one side and the radiation detector array 300 mounted on the other side. The radiation detector array 300 may have a curved shape along its long axis (i.e., the x-axis direction in FIG. 1A) such that each of the pixel detectors 126 along the length of the radiation detector may face towards the focal spot of the X-ray source 110. The gantry may also include a stationary (i.e., non-moving) part, such as a support, legs, mounting frame, etc., which rests on the floor and supports the moving part. The X-ray source 110 may emit a fan-shaped or cone-shaped X-ray beam 107 as the X-ray source 110 and the radiation detector array 300 rotate on the moving part of the gantry around the object 10 to be scanned. After the X-ray beam 107 is attenuated by the object 10, the X-ray beam 107 is received by the radiation detector array 300. The curved shape of the radiation detector array 300 may allow the CT imaging system 100 to most effectively reject radiation scattered by the object 10.

For each complete rotation of the X-ray source 110 and the radiation detector array 300 around the object 10, one cross-sectional slice of the object 10 may be acquired. As the X-ray source 110 and the radiation detector array 300 continue to rotate, the radiation detector array 300 may take numerous snapshots called “views”. Typically, about 1,000 profiles are taken in one rotation of the X-ray source 110 and the radiation detector array 300. The X-ray source 110 and the detector array 300 may slowly move relative to the object (e.g., patient) 10 along a horizontal direction (i.e., into and out of the page in FIG. 1A) so that the detector array 300 may capture incremental cross-sectional profiles over a region of interest (ROI) of the object 10, which may include the entire object 10. The data acquired by the radiation sensor(s) 80 and output by the read-out ASIC(s) 130 may be passed along to the computing device 160 that may be located remotely from the radiation detector array 300 via a connection 165. The connection 165 may be any type of wired or wireless connection. If the connection 165 is a wired connection, the connection 165 may include a slip ring electrical connection between any structure (e.g., gantry) supporting the radiation detector array 300 and a stationary support part of the support structure, which supports any part (e.g., a rotating ring). If the connection 165 is a wireless connection, the radiation detector array 300 may contain any suitable wireless transceiver to communicate data with another wireless transceiver that is in communication with the computing device 160. The computing device 160 may include processing and imaging applications that analyze each profile obtained by the radiation detector array 300, and a full set of profiles may be compiled to form a three-dimensional computed tomographic (CT) reconstruction of the object 10 and/or two-dimensional images of cross-sectional slices of the object 10.

Various alternatives to the design of the X-ray imaging system 100 of FIG. 1A may be employed to practice embodiments of the present disclosure. X-ray imaging systems may be designed in various architectures and configurations. For example, an X-ray imaging system may have a helical architecture. In a helical X-ray imaging scanner, the X-ray source 110 and radiation detector array 300 are attached to a freely rotating gantry. During a scan, the support structure (e.g., table) 105 moves the object 10 smoothly through the scanner, or alternatively, the X-ray source 110 and detector array 300 may move along the length of the object 10, creating helical path traced out by the X-ray beam. Alternatively, in an orbital X-ray imaging scanner, the object 10 may remain stationary while the gantry rotates completely or partially around the object 10 (e.g., to image a heart or a brain in one revolution). Slip rings may be used to transfer power and/or data on and off the rotating gantry. In other embodiments, the X-ray imaging system may be a tomosynthesis X-ray imaging system. In a tomosynthesis X-ray scanner, the gantry may move in a limited rotation angle (e.g., between 15 degrees and 60 degrees) in order to detect a cross-sectional slice of the object 10. The tomosynthesis X-ray scanner may be able to acquire slices at different depths and with different thicknesses that may be reconstructed via image processing. Alternatively, the X-Ray imaging system 100 may be a non-destructive testing system.

FIG. 1B illustrates components of an X-ray imaging system, including components within the ASIC 130 configured to count X-ray photons detected in each pixel detector 126 within a set of energy bins. As used herein, the terms “energy bin” and “bin” refer to a particular range of measured photon energies between a minimum energy threshold and a maximum energy threshold. For example, a first bin may refer to counts of photons determined to have an energy greater than a threshold energy (referred to as a trigger threshold, e.g., 20 keV) and less than 40 keV, while a second bin may refer to counts of photons determined to have an energy greater than 40 keV and less than 60 keV, and so forth.

X-rays 107 from an X-ray source (e.g., X-ray tube) 110 may be attenuated by a target (e.g., an object 10, such as a human or animal patient) before interacting with the radiation detector material within the pixelated detector array 300. An X-ray photon interacting (e.g., via the photoelectric effect) with a pixelated radiation detector material generates an electron cloud within the material that is swept by an electric field to the anode electrode 128. The charge gathered on the anode creates a signal that is integrated by a charge sensitive amplifier (CSA) 131. There may be a CSA 131 for each pixel detector 126 (e.g., for each anode 128) within the pixelated X-ray detector array 300. The voltage of the CSA output signal may be proportional to the energy of the X-ray photon. The output signal of the CSA may be processed by an analog filter or shaper 132.

The filtered output may be connected to the inputs of a number of analog comparators 134, with each comparator connected to a digital-to-analog converter (DAC) 133 that inputs to the comparator a DAC output voltage that corresponds to the threshold level defining the limits of an energy bin. The detector circuitry 130 may be configured so that after the CSA voltage has stabilized (after the dead time), that voltage may be between two voltage thresholds set by two DACs 133, which determines the output of the comparators 134. Outputs from the comparators 134 may be processed through decision gates 137, with a positive output from a comparator 134 corresponding to a particular energy bin (defined by the DAC output voltages) resulting in a count added to an associated counter 135 for the particular energy bin. Periodically, the counts in each energy bin counter 135 are output as signals 138 to the control unit 170.

Other suitable configurations for the read-out electronics of the ASIC 130 are within the contemplated scope of disclosure. For example, in some configurations, the analog voltage signals from the CSA may be converted to digital signals using an analog-to-digital converter (ADC) prior to being sorted into the respective energy bins.

The detector array 300 of an X-ray imaging system may include an array of radiation detector elements, referred to herein as pixel detectors. The signals from the pixel detectors may be processed by a pixel detector circuit (e.g., an above-described ASIC 130), which may sort detected photons into energy bins based on the energy of each photon or the voltage generated by the received photon. When an X-ray photon is detected, its energy is determined and the X-ray photon count for its associated energy bin is incremented. For example, if the detected energy of an X-ray photon is 24 kilo-electron-volts (keV), the X-ray photon count for the energy bin of 20-40 keV may be incremented. The number of energy bins may be three or more, such as four to twelve. In an illustrative example, an X-ray photon counting detector may have four energy bins: a first bin for detecting photons having an energy between 20 keV and 40 keV, a second bin for detecting photons having an energy between 40 keV and 60 keV, a third bin for detecting photons having an energy between 60 keV and 90 keV, and a fourth bin for detecting photons having an energy above 90 keV (e.g., between 90 keV and 120 keV). The greater the total number of energy bins, the better the material discrimination. The total number of energy bins and the energy range of each bin may be selectable by a user, such as by adjusting the threshold levels defining the limits of the respective energy bins in the read-out ASIC 130 as shown in FIG. 1B.

In various embodiments, a detector array 300 for an X-ray imaging system 100 as described above may include a plurality of pixel detectors 126 extending over a two-dimensional (2D) detector array surface. A typical radiation detector array 300 may include an array of individual radiation sensors 80 arranged side-by-side to provide the 2D detector array surface. The radiation sensors 80 may be located sufficiently close to each other to treat the 2D detector surface as essentially continuous, even though there are small gaps present between adjacent radiation sensors 80. Each radiation sensor 80 may comprise a semiconductor detector material plate 125, a continuous cathode electrode 122 located on a first side of the semiconductor detector material plate 125, and a plurality of anode electrodes 128 located on a second side of the semiconductor detector material plate 125. Each of the pixel detectors 126 comprises one the plurality of anode electrodes 128 and portions of the continuous cathode electrode 122 and the semiconductor detector material plate 125 overlying the one of the plurality of anode electrodes 128.

The detector array 300 (which is also referred herein as a detector module system (DMS)) may further include a modular configuration including a plurality of detector modules, where each detector module may include at least one above-described radiation sensor 80, at least one ASIC 130 (also known as a read-out integrated circuit (ROIC)) electrically coupled to the at least one radiation sensor 80, and a module circuit board. The module circuit board may support transmission of electrical power, control signals, and data signals between the module circuit board and the at least one ASIC 130 and the at least one radiation sensor 80 of the detector module, and may further support transmission of electrical power, control signals, and data signals between the module circuit board and the control unit 170 of the X-ray imaging system 100, other module circuit boards of the detector array, and/or a power supply for the detector array. A plurality of detector modules may be assembled on a common support structure, such as a detector array frame, to form a detector array 300.

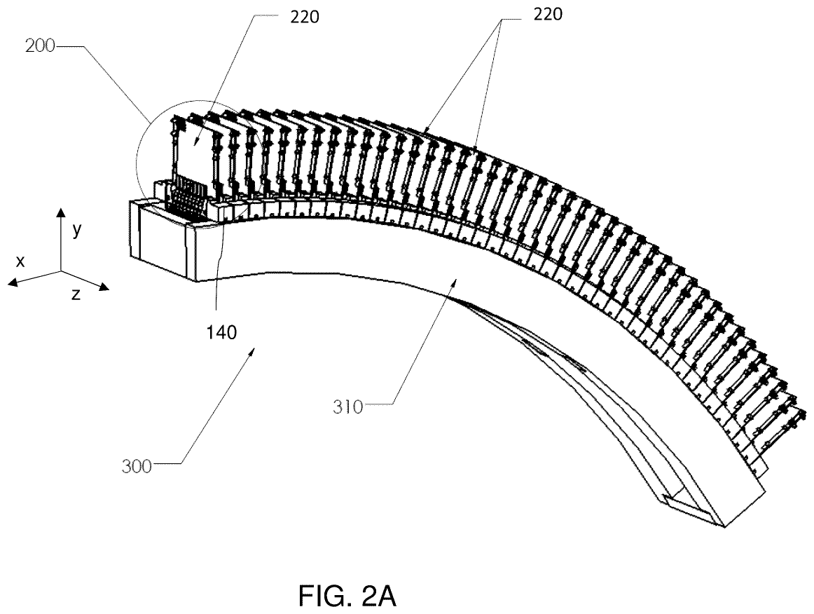

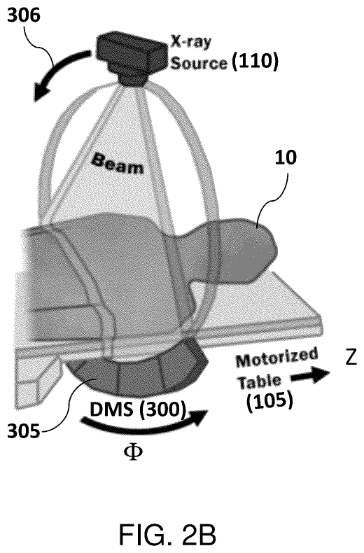

FIG. 2A is a rear perspective view of a detector array 300 for a computed tomography (CT) X-ray imaging system according to various embodiment of the present disclosure. The detector array 300 in this embodiment includes multiple detector modules 200 mounted on a detector array frame 310. The detector array frame 310 may be configured to provide attachment of a row of detector modules 200 such that physically exposed surfaces of the radiation sensors 80 of the detector modules 200 collectively form a curved detection surface located within a cylindrical surface. The multiple detector modules 200 may be assembled such that radiation sensors attached to neighboring detector modules 200 abut each other, i.e., make direct surface contact with each other and/or include a gap between adjacent radiation sensors that is less than 3 mm, and/or less than 2 mm, and/or less than 1 mm in the x-direction. In some embodiments, the detector modules 200 may be mounted to the detector array frame 310 by attaching frame bars 140 of the detector modules 200 to the detector array frame 310 using suitable mechanical fasteners. The radiation sensors and ASICs 130 of each module 200 may be mounted over a first (i.e., front) surface of the frame bar 140. Each module 200 may also include a module circuit board 220 extending away from a rear surface of the frame bar 140. Major surfaces of the module circuit boards 220 of the detector modules 200 may face each other in the detector array 300.

FIG. 2B is a perspective view of a CT X-ray imaging system 100 illustrating the orientation of the detector array 300 with respect to an X-ray source 110 and an object (e.g., patient) 10 being imaged according to various embodiments of the present disclosure. Referring to FIG. 2B, the X-ray source 110 and the detector array 300 (e.g., DMS) may rotate around the object 10 and the support structure (e.g., motorized table) 105 along the direction of arrow 306 to obtain cross-sectional image profiles (or “slices”) of the object 10. The X-ray source 110 and the detector array 300 may also be translated relative to the object 10 (e.g., by moving the support structure 105 and the object 10 with respect to the X-ray source 110 and the detector array 300 and/or by moving the X-ray source 110 and the detector array 300 along the length of the object 10) along a horizontal direction to obtain cross-sectional image “slices” of different portions of the object 10. The direction of the horizontal movement of the X-ray source 110 and the detector array 300 relative to the object 10 may be referred to as the “Z-axis” direction, which may be parallel to the axis of rotation of the X-ray source 110 and the detector array 300 around the object 10. As discussed above, the detector array 300 may also have a curved shape along the direction in which the X-ray source 110 and the detector array 300 rotate around the object 10. The pixel detectors 126 of the detector array 300 may be arranged in multiple columns and rows of pixel detectors, where each column may extend along the Z-axis direction, and each row may extend along the direction of rotation 306 (e.g., angular (D direction) of the detector array 300 around the object 10. Accordingly, the location of each pixel detector 126 within the detector array 300 may be defined by a unique row and column pair, where the location of the pixel detector 126 within a given column may be defined by its location along the Z-axis direction, and the location of the pixel detector within a given row may be defined by the azimuth angle D of a line segment extending between the pixel detector 126 and the focal spot of the X-ray source 110, where all pixel detectors 126 within the same column may have the same azimuth angle (D. The detector array 300 shown in FIG. 2B may be similar to the detector array 300 described above with reference to FIG. 2A. The detector array 300 may further include a suitable housing or enclosure 305 that encloses and protects the module circuit boards 220.

In some embodiments, each of the detector modules 200 of the detector array 300, such as the detector array 300 shown in FIGS. 2A and 2B may be constructed from a set of radiation detector units, which may also be referred to as “mini-modules” or “submodules.” In some embodiments, each of the radiation detector units may include one or more radiation sensors 80 coupled to one or more ASICs 130.

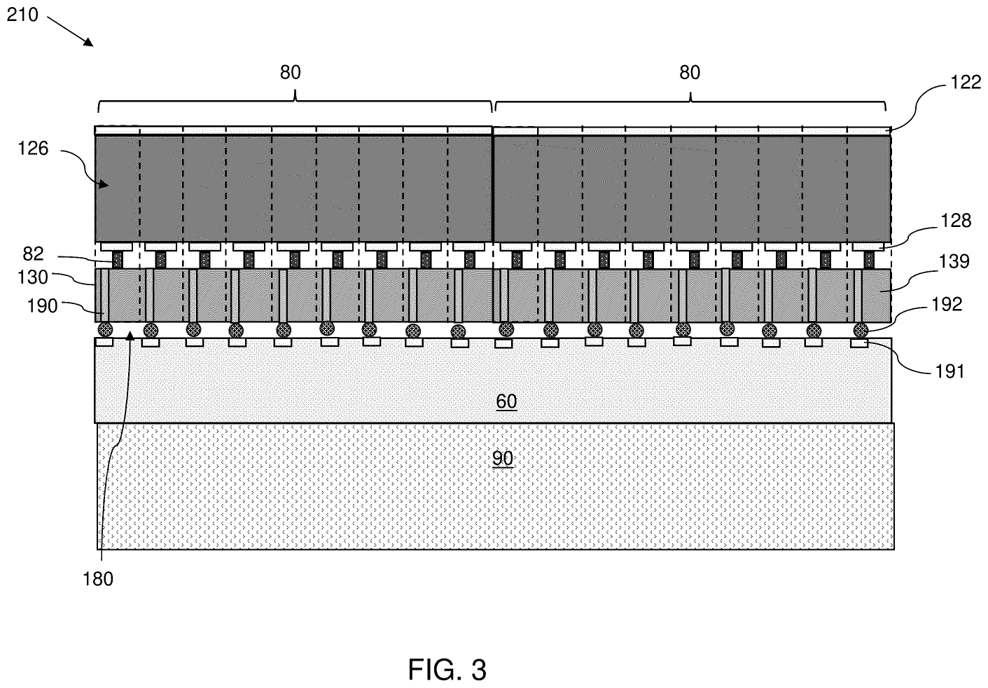

FIG. 3 is a vertical cross-sectional view of a radiation detector unit 210 according to one embodiment of the present disclosure. Referring to FIG. 3, the radiation detector unit 210 may include at least one radiation sensor 80 and at least one ASIC 130. Each radiation sensor 80 is directly mounted to an ASIC 130 via a plurality of bonding material portions 82. Each radiation sensor 80 may include an above-described detector material 125 having at least one cathode electrode 122 on a front side of the radiation sensor 80 and a plurality of anode electrodes 128 on a back side of the radiation sensor 80 defining an array of pixel detectors 126 as described above. As used herein, the “front side” of elements refers to the side that faces the incoming radiation, and the “backside” of elements refers to the side that is the opposite side of the front side.

Each radiation sensor 80 may be directly mounted to the front side of an ASIC 130 via a plurality of bonding material portions 82. In other words, each radiation sensor 80 may be mechanically and electrically coupled to an ASIC 130 via the plurality of bonding material portions 82, and no interposer or similar intervening structural component for routing of electrical signals between the radiation sensors 80 and the ASIC 130 is located between the back side of each of the radiation sensors 80 and the front side of an ASIC 130. Such a configuration may be referred to as a “direct attach” radiation detector unit 210. Exemplary embodiments of “direct attach” radiation detector units 210 and detector modules 200 are described, for example, in U.S. Provisional Patent Application No. 63/380,769, filed on Oct. 25, 2022, and U.S. patent application Ser. No. 18/158,695, filed on Jan. 24, 2023, the entire teachings of both of which are incorporated by reference herein for all purposes.

The plurality of bonding material portions 82 may be arranged in an array, such as a rectangular array, having the same periodicity as the periodicity of the anode electrodes 128 on the back sides of the radiation sensors 80. Thus, each bonding material portion 82 may electrically couple a respective anode electrode 128 of a radiation sensor 80 to the front side of an ASIC 130. In one non-limiting embodiment, the bonding material portions 82 may be composed of a conductive epoxy. Other suitable bonding materials, such as a low temperature solder material with under bump metallization, may be utilized to mount the radiation sensor 80 to the front side of an ASIC 130. An optional underfill material (not shown in FIG. 3) may be provided in the space between the back side surface of the radiation sensors 80 and the front side of the ASIC(s) 130 and laterally surrounding the bonding material portions 82. The underfill material may include a suitable insulating material, such as an insulating epoxy material.

FIG. 3 illustrates a pair of radiation sensors 80 mounted to the front side of a corresponding pair of ASICs 130. In some embodiments, the total number of radiation sensors 80 in the radiation detector unit 210 may be equal to the total number of ASICs 130 such that each radiation sensor 80 may be mounted to a corresponding ASIC 130. In other embodiments, the total number of radiation sensors 80 and the total number of ASICs 130 in the radiation detector unit 210 may not be equal. For example, a radiation detector unit 210 may include two or more radiation sensors 80 mounted to a single ASIC 130, and/or a single radiation sensor 80 may be mounted over the front sides of two or more separate ASICs 130. In some embodiments, the horizontal dimensions of the ASIC(s) 130 in the radiation detector unit 210 may be substantially equal to the combined dimensions of the radiation sensor(s) 80 mounted thereto along the corresponding horizontal directions. Thus, each pixel detector 126 of the radiation sensor(s) 80 may overlie a corresponding pixel region 180 of an ASIC 130. In some embodiments, the radiation detector unit 210 may have a rectangular periphery. This may enable any of the four peripheral sides of the radiation detector unit 210 to be abutted against a peripheral side of an adjacent radiation detector unit 210 upon assembly of multiple radiation detector units 210 in a detector array 300.

The radiation detector unit 210 of FIG. 3 may further include a carrier board 60 that is configured to route power supply to each of the ASICs 130 and the radiation sensors 80, control signals to the ASIC(s) 130, and data signals (e.g., digital detection signals) generated by the ASIC(s) 130. The carrier board 60 may be a circuit board including an insulating circuit board substrate and metal interconnect structures located on and/or within the substrate. In various embodiments, the ASIC(s) 130 may be disposed over the carrier board 60 such that the back side of each ASIC 130 may contact the front side of the carrier board 60.

In various embodiments, a plurality of through-substrate vias (TSVs) 190 may be provided in each of the ASICs 130. The TSVs 190 may include an electrically conductive material (e.g., a metal material, such as copper) that extends between the front side and the back side of the ASIC 130. In embodiments in which the ASIC 130 may be formed on and/or in a silicon substrate 139, the TSVs 190 may also be referred to as “through-silicon vias” which extend through the silicon substrate 139 of the ASIC 130 from the top to the bottom of the silicon substrate 139.

Accordingly, electrical connections between the carrier board 60 and each of the ASICs 130 may be made through the back side of the ASICs 130 via the plurality of TSVs 190. In particular, each of the TSVs 190 may be electrically connected to metal interconnect features 191 (e.g., bonding pads) located on the front side of the carrier board 60, as schematically illustrated in FIG. 3. This may obviate the need for wire bond and/or interposer connections between the front side of the carrier board 60 and the front side of each ASIC 130, which may help to minimize the footprint of the radiation detector unit 210. In some embodiments, the TSVs 190 may be connected to the metal interconnect features 191 on the carrier board 60 via a plurality of bonding material portions 192, which may include, for example, solder balls. In various embodiments, the outer periphery of the carrier board 60 may not extend beyond the outer periphery of the ASIC(s) 130 and the radiation sensor(s) 80 located over the carrier board 60 so as to provide a radiation detector unit 210 that is buttable on all four sides.

The radiation sensor(s) 80, the ASIC(s) 130 and the carrier board 60 may be mounted to a supporting substrate (e.g., a block) 90 as shown in FIG. 3. The supporting substrate 90 may include a high thermal conductivity material such as a metal (e.g., aluminum, copper, etc.). The metal of the supporting substrate may have a coefficient of thermal expansion that is matched or closely-matched to (e.g., within 10 percent of) to that of the carrier board 60 and/or other constituent parts of the radiation detector unit 210. The supporting substrate 90 may function as a heat sink for the radiation detector unit 210. The supporting substrate 90 may be attached to the backside of the carrier board 60 using a thermally conductive adhesive such as a thermally conductive paste, and/or by mechanical connection structures (such as snap-in connectors, screws, and/or bolts and nuts).

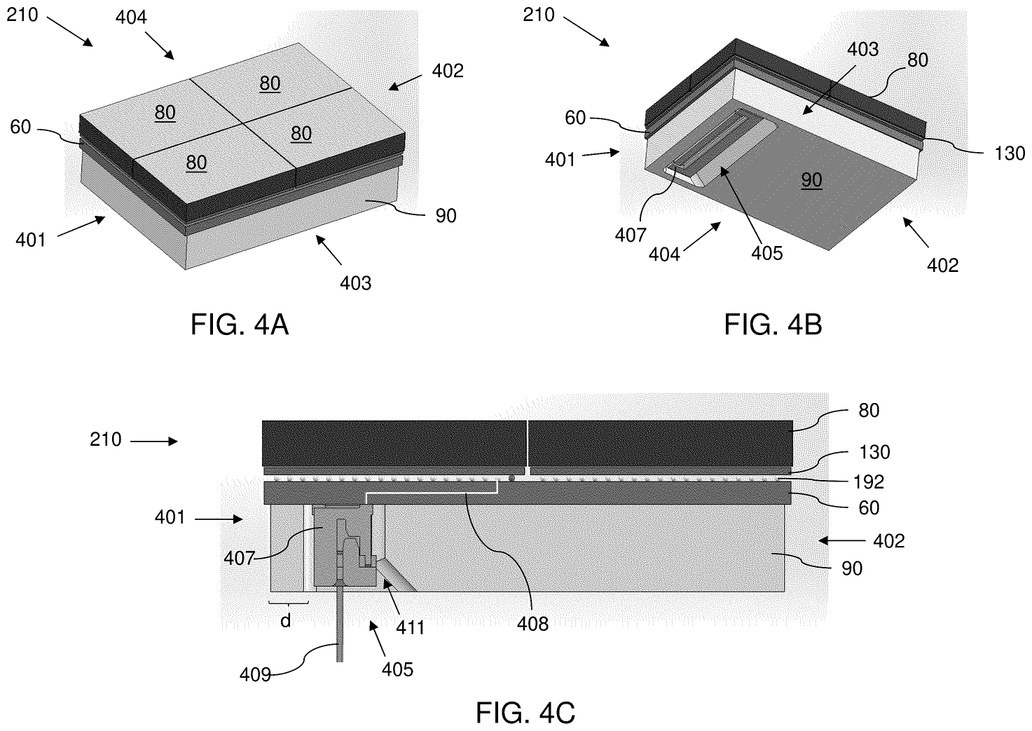

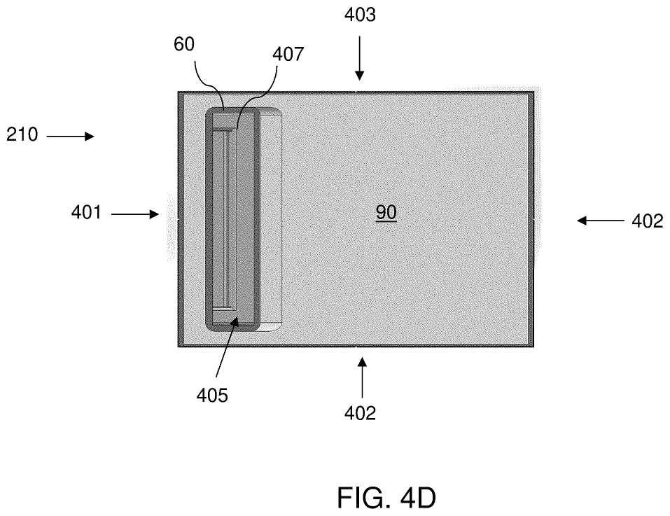

FIG. 4A is a top perspective view of a radiation detector unit 210 according to various embodiments of the present disclosure. FIG. 4B is a bottom perspective view of the radiation detector unit 210 of FIG. 4A. FIG. 4C is a side cross-section view of the radiation detector unit 210 of FIGS. 4A and 4B. FIG. 4D is a bottom view of the radiation detector unit 210 of FIGS. 4A-4C. Referring to FIGS. 4A-4D, the radiation detector unit 210 in this embodiment includes a 2×2 array of radiation sensors 80 and ASICs 130 mounted to a common carrier board 60 and a common supporting substrate 90. The radiation detector unit 210 may have a rectangular horizontal cross-sectional shape, with a first side 401, a second side 401 opposite the first side 401, a third side 403, and a fourth side 404 opposite the third side 403. The first and second sides 401, 402 of the radiation detector unit 210 may form the shorter sides of the rectangle, and the third and fourth sides 403, 404 may form the longer sides of the rectangle. Other suitable shapes and configurations of the radiation detector unit 210 are within the contemplated scope of the disclosure. The interior edges of the radiation sensors 80 (i.e., the edges of the radiation sensors 80 that do not form the exterior sides 401, 402, 403 and 404 of the radiation detector unit 210) may abut one another such that the radiation sensors 80 may form a continuous detector surface.

The supporting substrate 90 may include an opening 405 though the supporting substrate 90. The opening 405 may be laterally surrounded on all sides by the high thermal conductivity material of the supporting substrate 90. The opening 405 may have a generally rectangular shape and may extend parallel to the “short” sides of the radiation detector unit (i.e., sides 401 and 402). Other suitable opening 405 shapes (e.g., circular, oval, etc.) may be used instead. A connector 407 may be located within the opening 405. The connector 407 may be configured to electrically connect the carrier board 60 of the radiation detector unit 210 to one or more cables 409. In some embodiments, the one or more cables 409 may include a flexible flat cable (FFC), which may also be referred to as a “ribbon connector.” In some embodiments, the one or more cables 409 may include a flat printed cable (FPC) (also referred to as a flexible printed circuit board), which is a type of FFC in which the conductors are printed on the cable substrate instead of being embedded within it. In some embodiments, the connector 407 may include a receptacle into which the one or more cables 409 (e.g., an FFC) may be inserted. The connector 407 may also include a locking feature, such as flip lock or a slide lock, that may be actuated to lock the cable 409 within the connector 407. In some embodiments, the opening 405 in the supporting substrate 90 may include a relief feature 411 as shown in FIG. 4C that permits the locking feature of the connector 407 to be accessed to lock and/or release the cable 409 inserted into the connector 407. Other suitable configurations for the connector 407 and cable(s) 409 are within the contemplated scope of disclosure.

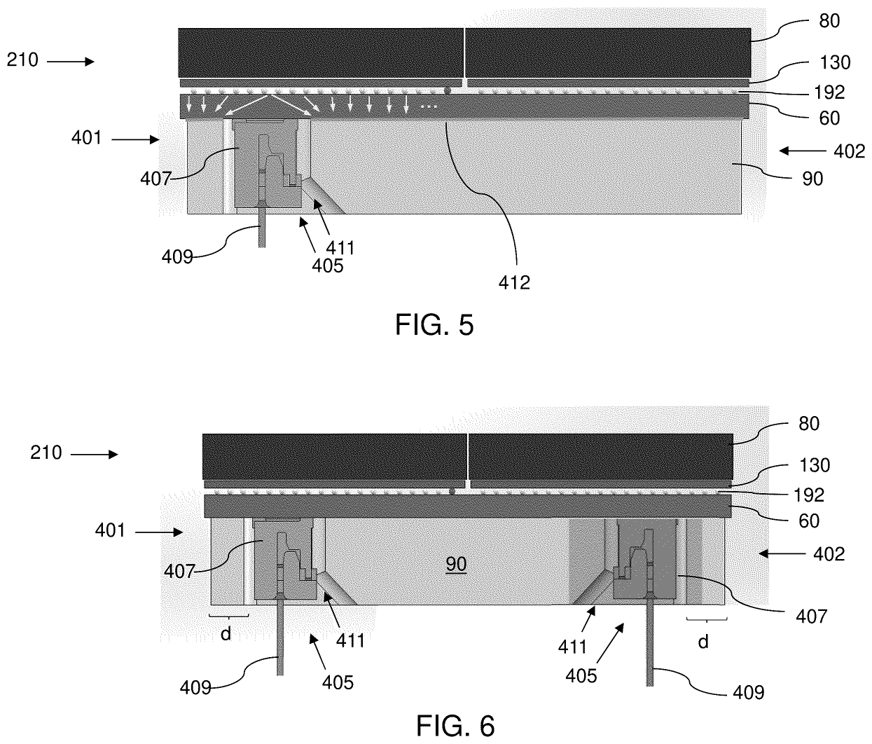

The carrier board 60 may include conductive interconnect features 408, such as metal lines and vias, located within the carrier board 60 that may be used to transmit power and data signals between the ASIC(s) 130 and the connector 407. In some embodiments, a portion of the back side of the carrier board 60 may be exposed within the opening 405 around the periphery of the connector 407. Alternatively, or in addition, portions of a thermally-conductive heat dissipator 412 (described below with respect to FIG. 5) that is located between the carrier board 60 and the supporting substrate 90 may be exposed within the opening 405 around the periphery of the connector 407. In this embodiment, the thermally-conductive heat dissipator 412 may comprise a carbon based material, such as pyrolytic carbon.

Accordingly, the cable(s) 409 and the connector 407 may be used to route power supply to the ASIC(s) 130 and to the radiation sensor(s) 80, control signals to the ASIC(s) 130, and data signals (e.g., digital detection signals) generated by the ASIC(s) 130 and transmitted through the carrier board 60. One end of the each cable 409 may be attached to the connector 407, and the other end of the cable 409 may be connected to a module circuit board 220 as shown in FIG. 2A. Although a single opening 405 and connector 407 is shown in the embodiment of FIGS. 4A-4D, it will be understood a radiation detector unit 210 may include multiple connectors 407 located within one or more openings 405 through the back side of the supporting substrate 90.

In various embodiments, by providing the electrical connection to the radiation detector module 210 through the back side of the supporting substrate 90, space may be conserved around the sides 401, 402, 403 and 404 of the radiation detector unit 210. Accordingly, any of the four sides 401, 402, 403 and 404 of the radiation detector unit 210 may be abutted against the side of a neighboring radiation detector unit 210 without needing to accommodate any electrical connections to the side(s) of the radiation detector unit 210. This may help to facilitate the assembly of a large area detector array.

A potential drawback of providing the electrical connection to the radiation detector module 210 through the back side of the supporting substrate 90 is that this may result in thermal non-uniformities within the radiation detector module 210. In particular, a lower amount of heat transfer in the region of the radiation detector unit 210 located above the opening 405 and the connector 407 may result in relatively less heat transfer and the generation of a thermal “hot spot” in this region. In various embodiments, the opening 405 and the connector 407 may be offset to one side of the radiation detector unit 210 (e.g., closer to side 401 than to the opposite side 402). This may provide sufficient space for a heat sink to be positioned below the central region of the radiation detector unit 210, as described in further detail below.

Referring to FIG. 4C, the opening 405 may be spaced from the peripheral edge of the detector (i.e., the edge defined by side 401 in FIG. 4C) by a minimum distance, d. In various embodiments, the minimum distance, d, may be sufficient such that the thermal resistance on opposite sides of the opening 405 (i.e., to the left and right of the opening in FIG. 4C) may be roughly equivalent. Locating the opening 405 too close to the side 401 of the radiation detector unit 210 may result in the formation of a hot spot along the edge of the radiation detector unit 210. However, by spacing the opening 405 away from the side 401 of the radiation detector unit 210, heat that is generated above the opening 405 and the connector 407 may travel through the carrier board 60 to both the left and right sides of the opening 405 and into the thermally-conductive material of the supporting substrate 90 on either side of the opening 405. In some embodiments, the minimum distance, d, may be at least about 1.5 mm, such as at least about 2 mm.

FIG. 5 is a side cross-section view of a radiation detector unit 210 according to another embodiment of the present disclosure. Referring to FIG. 5, the radiation detector unit 210 may include a heat dissipator 412 located between the front side of the carrier board 60 and the front side of the supporting substrate 90 (e.g., either embedded in the carrier board 60 or located over the backside of the carrier board 60). The heat dissipator 412 may include a thin sheet or plate of a thermally-conductive material, such as copper or carbon. The arrows in FIG. 5 schematically illustrate the thermal paths from the radiation sensors 80 and ASICs 130 through the carrier board 60 to the supporting substrate 90 and the heat dissipator 412. The heat dissipator 412 may help to spread the heat laterally within the radiation detector unit 210 and to couple the heat generated by the radiation sensors 80 and ASICs 130 into the supporting substrate 90.

FIG. 6 is a side cross-section view of a radiation detector unit 210 including two connectors 407 according to another embodiment of the present disclosure. The radiation detector unit 210 shown in FIG. 6 may be similar to the radiation detector unit 210 described above with reference to FIGS. 4A-4D. Thus, repeated discussion of like elements is omitted for brevity. The radiation detector unit 210 of FIG. 6 may additionally include a second opening 405 through the supporting substrate 90 and a second connector 407 located within the second opening 405. A cable 409 may be inserted into the second connector 407. The second connector 407 may be offset towards the opposite side of the radiation detector unit 210 from the first connector 407. The second opening 405 may be spaced away from the second side 402 of the radiation detector unit 210 by a minimum distance, d, of at least about 1.5 mm, such as at least about 2 mm.

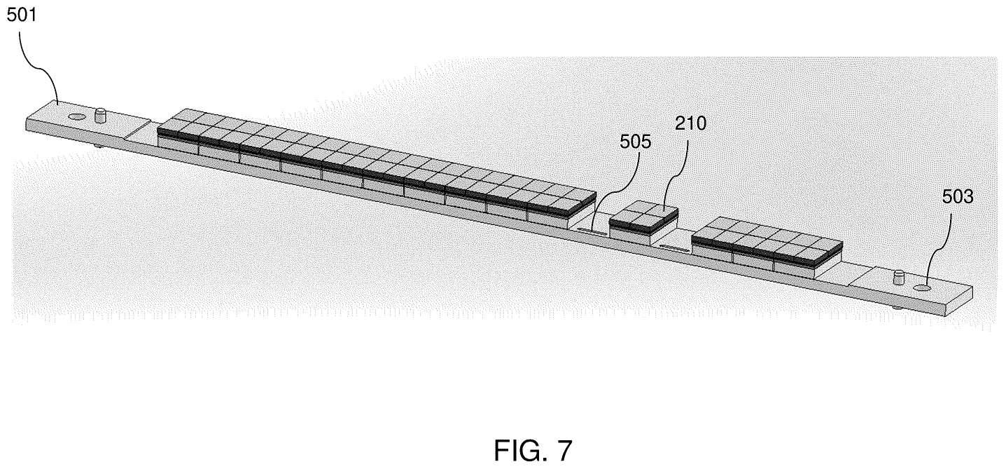

FIG. 7 is a perspective view illustrating a plurality of radiation detector units 210 mounted on a frame bar 501 according to various embodiments of the present disclosure. Referring to FIG. 7, a column of radiation detector units 210 may be mounted adjacent to one another on the front side of the frame bar 501. Engagement features (not shown in FIG. 7) may optionally be provided on the front side of the frame bar 501 that may mate with corresponding engagement features (not shown) on the backside of the supporting substrate 90 of each of the radiation detector units 210. Mounting features 503 on the ends of the frame bar 501 may be used to assemble the frame bar 501 and the radiation detector units 210 into an above-described detector module 200 and/or a detector array 300 (e.g., DMS). The frame bar 501 may include slots 505 that may be sized and shaped to permit the cables 409 (see FIG. 4C) of each radiation detector unit 210 to pass through the slots 505. In the embodiment shown in FIG. 7, sixteen (16) radiation detector units 210, each including a 2×2 array of radiation sensors 80, may be mounted in a column along the front side of the frame bar 501. It will be understood that other configurations for mounting the radiation detector units 210 to a frame bar 501 are within the scope of this disclosure.

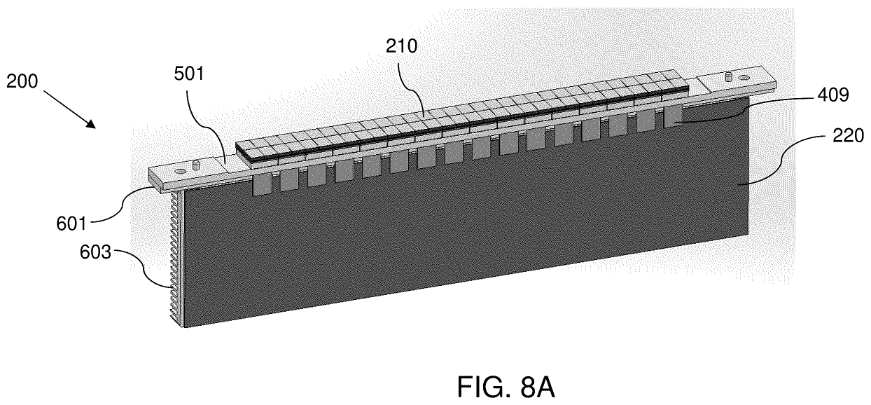

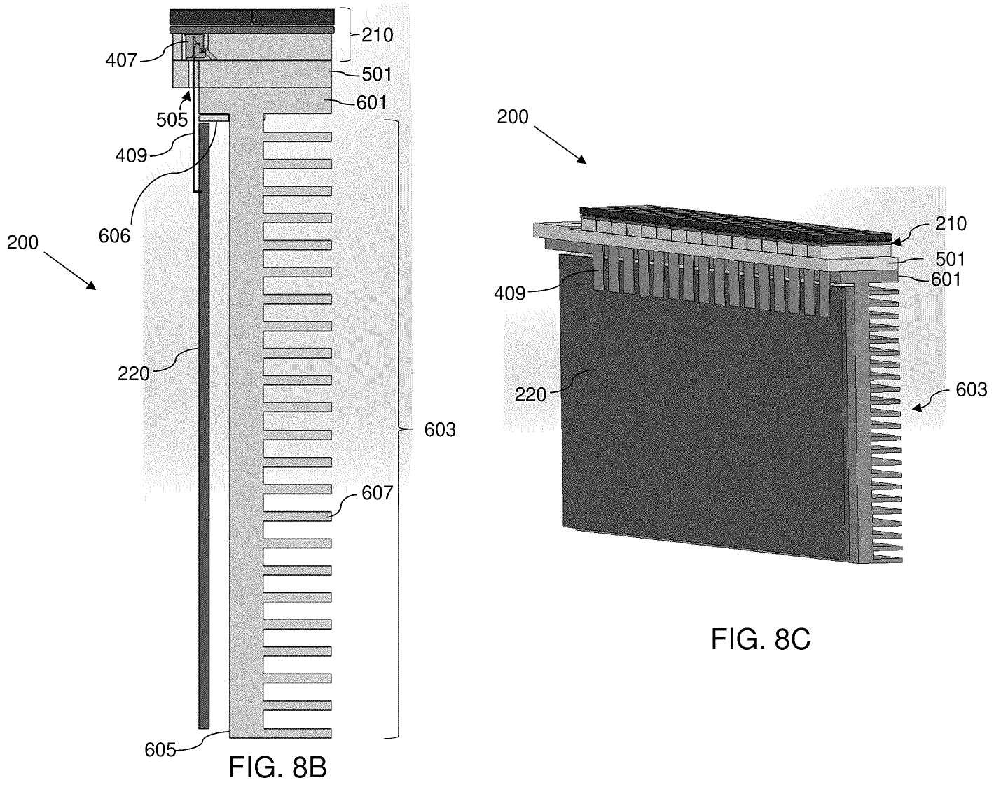

FIG. 8A is a perspective view of a detector module 200 according to an embodiment of the present disclosure. FIG. 8B is a side cross-section view of the detector module 200 of FIG. 8A. FIG. 8C is a perspective cross-section view of the detector module 200 of FIGS. 8A and 8B. Referring to FIGS. 8A-8C, a frame bar 501 having a plurality of radiation detector units 210 as shown in FIG. 7 may be mounted to the front side of a lower plate 601 via a suitable adhesive and/or mechanical fastening elements (not shown in FIGS. 8A-8C). A heat sink, such as an air cooled heat sink, 603 may extend from the back side of the lower plate 601 away from the frame bar 501. In some embodiments, the lower plate 601 and the heat sink 603 may include an integral structure composed of a suitable thermally-conductive material, such as aluminum or copper. In other embodiments, the lower plate 601 and the heat sink 603 may include a multi-component assembly. The detector module 200 may also include an above-described module circuit board 220. The module circuit board 220 may be attached to the lower plate 601 and/or the heat sink 603 via suitable mechanical fasteners (not shown in FIGS. 8A-8C). The module circuit board 220 may extend away from the frame bar 501 parallel to the heat sink 603.

As shown in FIG. 8B, the cables 409 may extend from the connectors 407 of each radiation detector unit 210 through the respective slots 505 in the frame bar 501 and may be electrically connected to the module circuit board 200. The width of the lower plate 601 may be less than the width of the frame bar 501 to provide sufficient clearance for the cables 409 to pass along the side surface of the lower plate 601. A radiation shield 606, which may include tungsten, lead, or a similar X-ray attenuating material, may be located between the lower surface of the lower plate 601 and the top edge of the module circuit board 220 to provide X-ray shielding for the module circuit board 220.

In various embodiments, the heat sink 603 of the detector module 220 may be a fin-type heat sink, such as a fin-type air cooled heat sink. Alternatively, or in addition, the heat sink 603 may include a different type of heat sink, such as a heat exchanger that includes one or more heat pipes. As shown in FIG. 8B, a fin-type heat sink 603 may include a plate member 605 that may extend away from the frame bar 501 parallel to the module circuit board 220. The plate member 605 may underlie a central region of each of the radiation detector units 210 of the detector module 200. A plurality of fins 607 may extend from the plate member 605 in a direction perpendicular to the plate member 605 and to the major surfaces of the module circuit board 220 and parallel to the front sides of the radiation sensors 80 of the radiation detector units 210. In various embodiments, the fins 607 may extend away from the module circuit board 220 and may extend towards, but may not extend beyond, the second sides 402 of the radiation detector modules 220. This may permit a plurality of detector modules 220 to be abutted along the <D direction as shown in FIG. 2B to provide the detector array 300.

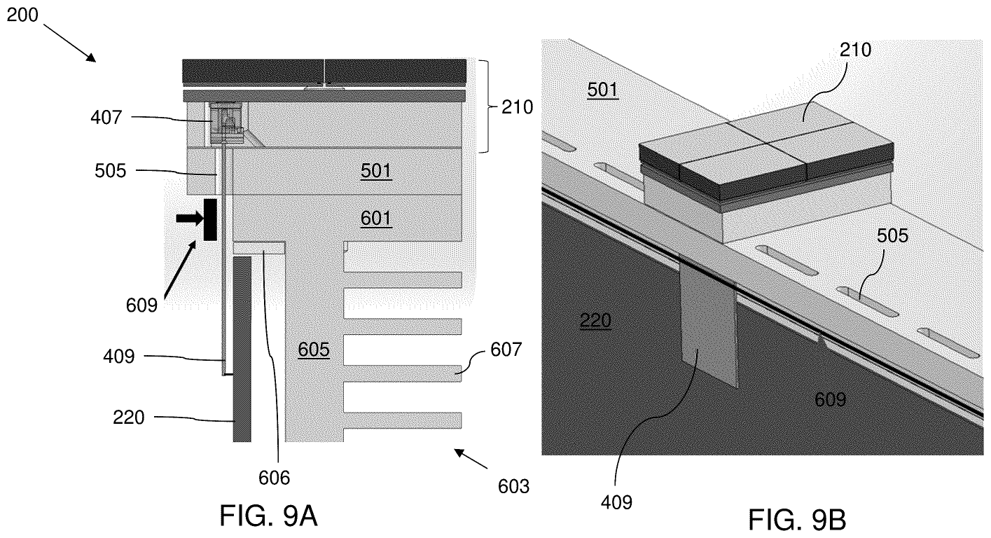

FIG. 9A is a side cross-section view of a portion of a detector module 200 according to another embodiment of the present disclosure. FIG. 9B is a perspective view of the detector module 200 of FIG. 9A. The detector module shown in FIGS. 9A and 9B may be similar to the detector module 200 described above with reference to FIGS. 8A-8C. Thus, repeated discussion of like elements is omitted for brevity. The detector module 200 of FIGS. 9A and 9B may additionally include a retention bar 609 that may be pressed toward the side surface of the lower plate 601 (as indicated by the arrow in FIG. 9A) to clamp the cable 409 against the lower plate 601. In such a configuration, the connectors 407 that connect the cables 409 between the respective radiation detector units 210 and the module circuit board 220 need not have locking features, or if they have locking features, need not have them engaged.

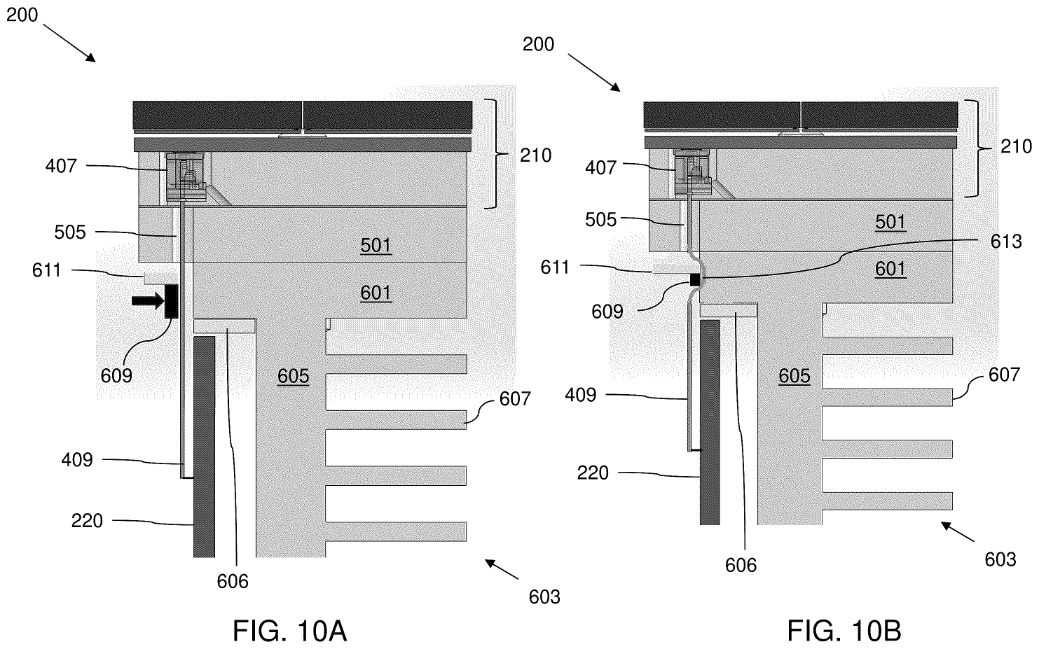

FIG. 10A is a side cross-section view of a portion of a detector module 200 according to another embodiment of the present disclosure. The detector module shown in FIG. 10A may be similar to the detector module 200 described above with reference to FIGS. 9A and 9B. In the embodiment of FIG. 10A, the retention bar 609 may further include a radiation shield component 611 including a suitable X-ray absorbent material (e.g., tungsten, etc.) that may provide enhanced radiation shielding for the module circuit board 220. FIG. 10B is side cross-section view of a variant of the detector module of FIG. 10A in which there is a jog 613 in the path of the cables 409 to eliminate any gap in the radiation shielding provided by the radiation shield 606 and the radiation shield component 611 of the retention bar 609.

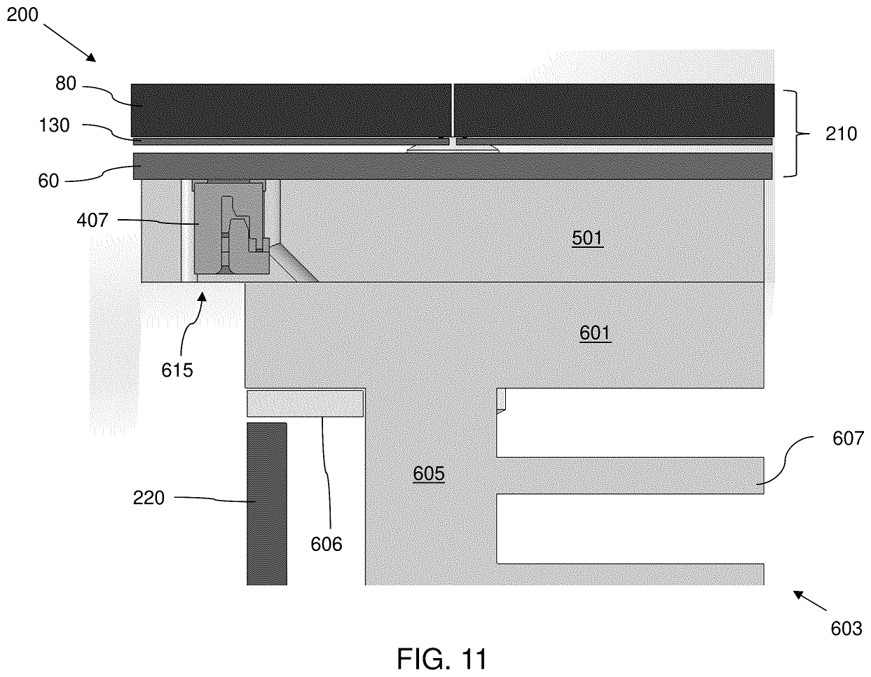

FIG. 11 is a side cross-section view of a portion of a detector module 200 according to yet another embodiment of the present disclosure. Referring to FIG. 11, the radiation detector units 210 of the detector module 200 may each include at least one radiation sensor 80 and at least one ASIC 130 mounted to a carrier board 60, as described above. The detector module 200 of FIG. 11 may differ from the previously-described embodiments in that the carrier boards 60 of the radiation detector units 210 may be directly mounted to the front side of the frame bar 501. That is, the frame bar 501 functions as the common supporting substrate for plural detector units 210, and each of the radiation detector units 210 may lack an individual, dedicated supporting substrate (e.g., metal block) 90 as described above. In some embodiments, the frame bar 501 of the detector module 200 may include a plurality of openings 615 with above-described connectors 407 located within each of the openings 615. The connectors 407 and cables (not shown in FIG. 11) inserted within the connectors 407 may provide the electrical connections between each of the radiation detector units 210 and the module circuit board 220. In some embodiments, the carrier boards 60 of the radiation detector units 210 may be mounted to the frame bar 501 using a suitable adhesive. In some embodiments, the adhesive may be designed to lose its adhesive properties when subjected to heat or certain types of radiation (e.g., UV radiation), which may allow the radiation detector units 210 to be removed from the frame bar 501 for servicing, replacement, or rework. Other suitable methods for mounting the radiation detector units 210 to the frame bar 501 may also be utilized.

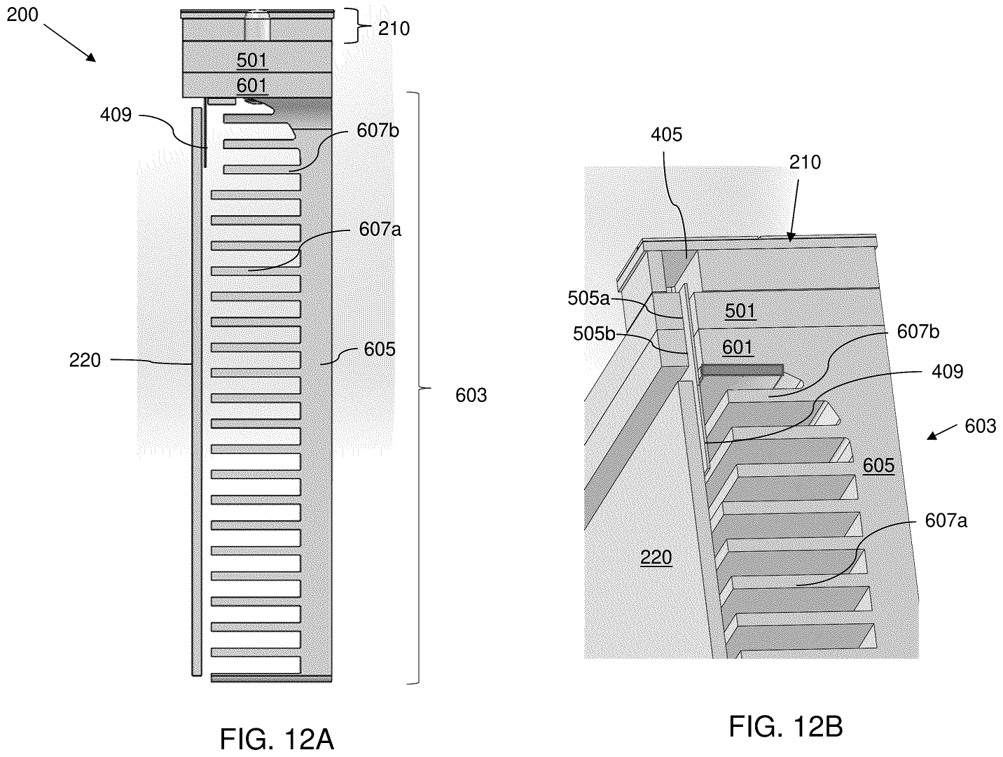

FIG. 12A is a side cross-section view of a portion of a detector module 200 according to another embodiment of the present disclosure. FIG. 12B is a perspective view of a portion of the detector module 200 of FIG. 12A. In the detector module 200 of FIGS. 12A and 12B, the module circuit board 220 and the plate member 605 of the heat sink 603 may be located on opposite sides of the detector module 200, with the fins 607a, 607b of the heat sink 603 extending from the plate member 605 towards the module circuit board 220. This may enable at least some of the fins 607a to have a longer length in comparison to the fins 607 in the embodiments shown in FIGS. 8A-11.

As shown in FIG. 12B, the cables 409 may pass through a pair of slots 505a and 505b extending through the frame bar 501 and the lower plate 601, respectively, to connect the radiation detector units 210 to the module circuit board 220. The fins of the heat sink 603 may have non-uniform lengths, with a first set of fins 607a having a relatively longer length and a second set of fins 607b that may have a shorter length to accommodate the cables 409. In some embodiments, the lengths of the fins 607b may vary along the direction extending into and out of the page in FIGS. 12A and 12B, where the fins 607b may be shorter in regions in which the cables 409 connect to the module circuit board 220 and longer in the regions between the cables 409. The cables 409 may connect to the side of the module circuit board 220 that faces towards the heat sink 603.

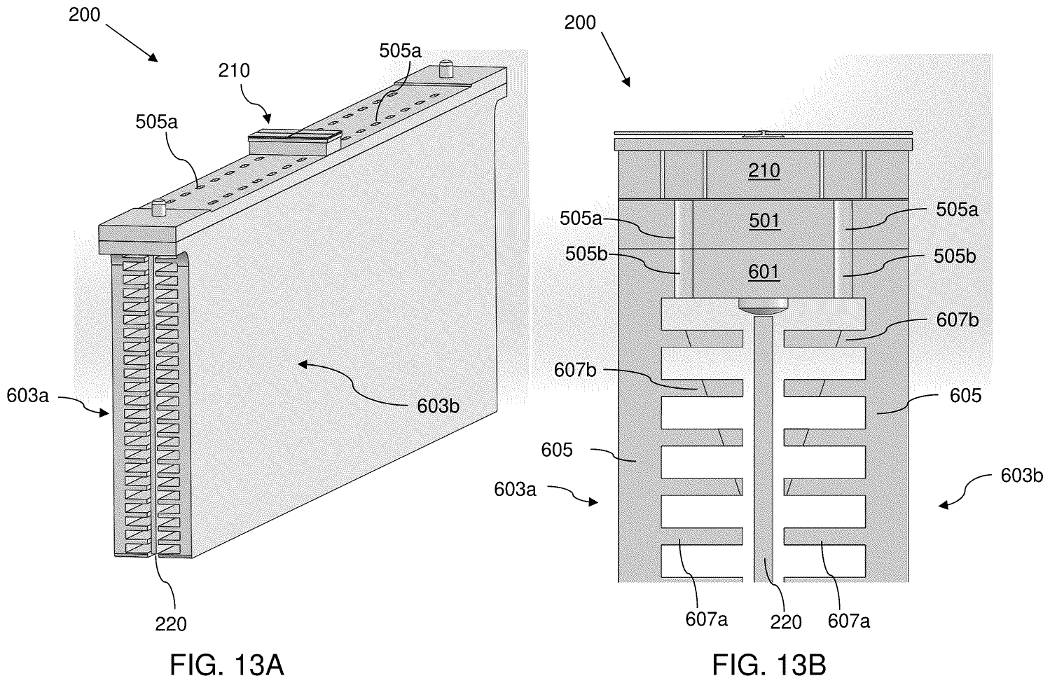

FIG. 13A is a perspective view of a detector module 200 according to another embodiment of the present disclosure. FIG. 13B is a side cross-section view of a portion of the detector module 200 of FIG. 13A. Referring to FIGS. 13A and 13B, the detector module 200 may include a centrally-located module circuit board 220 with heat sinks 603a and 603b located on either side of the module circuit board 220. Each of the radiation detector units 210 may include a pair of connectors 407 as shown in the embodiment of FIG. 6. A pair of cables 409 may pass through respective slots 505a and 505b extending through the frame bar 501 and the lower plate 601 to connect the pair of connectors 407 (e.g., as shown in FIG. 6) in the radiation detector units 210 to opposite sides of the module circuit board 220. Each heat sink 603a and 603b may include a plate member 605 and a plurality of fins 607a, 607b extending from the plate member 605 towards the centrally-located module circuit board 220. As in the embodiment of FIGS. 12A and 12B, the fins of the heat sinks 603a and 603b may have non-uniform lengths, with a first set of fins 607a having a relatively longer length and a second set of fins 607b that may have a shorter length to accommodate the cables 409. In some embodiments, the lengths of the fins 607b may vary along the direction into and out of the page in FIG. 13B), where the fins 607b may be shorter in regions in which the cables 409 connect to the module circuit board 220 and longer in the regions between the cables 409.

The devices of the embodiments of the present disclosure can be employed in various radiation detection systems including computed tomography (CT) imaging systems. Any direct conversion radiation sensors may be employed such as radiation sensors employing Si, Ge, GaAs, CdTe, CdZnTe, and/or other similar semiconductor materials.

The radiation detectors of the present embodiments may be used for medical imaging as radiation detectors in High-Flux applications as in X-ray Computed Tomography (CT) for medical applications, and for non-medical imaging applications, such as in baggage security scanning and industrial inspection applications.

While the disclosure has been described in terms of specific embodiments, it is evident in view of the foregoing description that numerous alternatives, modifications and variations will be apparent to those skilled in the art. Each of the embodiments described herein can be implemented individually or in combination with any other embodiment unless expressly stated otherwise or clearly incompatible. Accordingly, the disclosure is intended to encompass all such alternatives, modifications and variations which fall within the scope and spirit of the disclosure and the following claims.

1. A detector structure, comprising:
a supporting substrate having an opening therethrough that is laterally surrounded on all sides by the supporting substrate;
a carrier board located over a front side of the supporting substrate, the carrier board comprising interconnect structures electrically extending between the front side and a back side of the carrier board;
at least one application specific integrated circuit (ASIC) located over the carrier board, the at least one ASIC comprising signal processing channel circuitry;
at least one radiation sensor located over a front side of the at least one ASIC; and
a connector located within the opening in the supporting substrate, wherein the connector is electrically coupled to the interconnect structures on the back side of the carrier board.

2. The detector structure of claim 1, wherein:
the at least one radiation sensor comprises an array of pixel detectors that generate event detection signals in response to photon interaction events occurring within the pixel detectors;
the signal processing channel circuitry of the at least one ASIC is configured to convert the event detection signals from each of the pixel detectors into digital detection signals;
the at least one ASIC comprises a plurality of through-substrate vias extending through an ASIC substrate and electrically coupled to the signal processing channel circuitry located on a front side of the ASIC substrate; and
the interconnect structures are electrically coupled to the plurality of through-substrate vias.

3. The detector structure of claim 1, wherein the detector structure comprises a plurality of ASICs and a plurality of radiation sensors mounted over the front sides of the corresponding ASICs.

4. The detector structure of claim 1, further comprising a thermally-conductive heat dissipator located between the front side of the carrier board and the front side of the supporting substrate.

5. The detector structure of claim 1, wherein:
the detector structure has a rectangular horizontal cross-section shape, and the opening and the connector are offset towards a first side of the detector structure; and
a distance between the first side and the opening is 1.5 mm or more.

6. The detector structure of claim 5, wherein:
the opening comprises a first opening and the connector comprises a first connector;
the supporting substrate has a second opening through the supporting substrate and laterally surrounded on all sides by the supporting substrate;
a second connector is located within the second opening in the supporting substrate;
the second connector is electrically coupled to the interconnect structures on the back side of the carrier board;
the second opening and the second connector are offset towards a second side of the detector structure opposite the first side; and
a distance between the second side and the second opening is 1.5 mm or more.

7. The detector structure of claim 1, wherein the at least one radiation sensor comprises cadmium zinc telluride, and the supporting substrate comprises a metal block.

8. The detector structure of claim 1, further comprising a cable coupled to the connector in the opening through the supporting substrate.

9. The detector structure of claim 8, wherein:
the cable comprises a flexible flat cable or flexible printed circuit board; and
the opening comprises a relief feature to permit actuation of a locking feature on the connector to secure the cable within the connector.

10. A detector module, comprising:
the detector structure of claim 8;
the heat sink extending away from the rear side of the detector structure; and
a module circuit board extending away from a rear side of the detector structure and electrically connected to the cable.

11. The detector module of claim 10, further comprising a frame bar.

12. The detector module of claim 10, wherein:
a column of the radiation detector structures is mounted on a front side of the frame bar;
the module circuit board extends away from a rear side of the frame bar;
a plurality of cables extend between the respective connector of each radiation detector structures and the module circuit board; and
the heat sink extends away from the rear side of the frame bar.

13. The detector module of claim 12, further comprising a lower plate, wherein the frame bar is mounted over the front side of the lower plate, and the module circuit board and the heat sink extend away from a back side of the lower plate.

14. The detector module of claim 13, wherein the heat sink and the lower plate comprise an integral structure.

15. The detector of claim 13, further comprising a plurality of slots through the frame bar, wherein each of the cables extends from the respective connector through the respective slot through the frame bar and is connected to the module circuit board.

16. The detector module of claim 13, wherein the heat sink comprises an air cooled heat sink comprising a plate member that extends away from the back side of the lower plate parallel to the module circuit board, and a plurality of fins that extend from the plate member.

17. The detector module of claim 13, further comprising:
a radiation shield located between the lower plate and the module circuit board; and
a retention bar configured to clamp each of the plurality of cables against a side surface of the lower plate, wherein the retention bar comprises a radiation shielding component.

18. The detector module of claim 10, wherein:
the supporting substrate comprises portion of a frame bar;
the frame bar comprises a plurality of openings through the frame bar, and
a plurality of the connectors are located within the respective openings through the frame bar.

19. An X-ray imaging system, comprising:
a radiation source configured to emit X-rays; and
a detector array including a plurality of detector modules of claim 10 that form a continuous detector surface and that are configured to receive the X-rays from the radiation source through an intervening space configured to contain an object therein.

20. A method of fabricating a detector module, comprising:
mounting a column of radiation detector units over a front side of a frame bar comprising a plurality of slots, wherein each radiation detector unit comprises a carrier board, at least one application specific integrated circuit (ASIC) located over a front side of the carrier board, and at least one radiation sensor located over the at least one ASIC, and a connector electrically coupled to interconnect structures on the back side of the carrier board;
providing a module circuit board and a heat sink extending away from a rear side of the frame bar; and
connecting a plurality of cables between the connector of each radiation detector unit and the module circuit board through the plurality of slots in the frame bar.
